(19) United States
(12) Patent Application Publication
(10) Pub. No.: US 2007/0238137 A1
(43) Pub. Date: Oct. 11, 2007

(54) CONSTITUTIVELY RESISTANT CANCER STEM CELLS IN DIAGNOSIS

(75) Inventors: Vera Svobodova Donnenberg, Pittsburgh, PA (US); Albert David Donnenberg, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh - Of the Commonwealth System of Higher Education, Pittsburgh, PA (US)

(21) Appl. No.: 11/733,050

(22) Filed: Apr. 9, 2007

Related U.S. Application Data
(60) Provisional application No. 60/790,324, filed on Apr. 7, 2006.
(60) Provisional application No. 60/801,292, filed on May 18, 2006.

(51) Int. Cl. G01N 33/574

(52) U.S. Cl. 435/7.23

(57) ABSTRACT

The invention provides a method of identifying circulating clonogenic cancerous cells, specifically multiply-drug resistant (MDR) cancer stem cells.

### CROSS-REFERENCE TO RELATED APPLICATIONS

This application claims priority to U.S. Provisional Patent Application 60/790,324, filed Apr. 7, 2006. This application also claims priority to U.S. Provisional Patent Application 60/801,292, filed May 18, 2006. The contents of these priority applications are incorporated herein in their entirety.

### STATEMENT CONCERNING FEDERALLY SPONSORED RESEARCH

Research leading to this invention was funded, in part, through grants from the United States Department of Defense under award numbers BC044784, and BC032981. The Government of the United States of America may have certain rights in this invention.

### BACKGROUND OF THE INVENTION

The ability to identify circulating tumor cells has afforded the ability, in some instances, to diagnose certain cancers and has led to a better understanding of the biological mechanisms of cancer pathogenesis. Few technologies for identifying circulating cancer cells can be used clinically. For example, the CELLSEARCH™ Circulating Tumor Cell Kit is intended for the enumeration of circulating tumor cells of epithelial origin (CD45−, EpCAM+, and cytokeratins 8, 18+, and/or 19+) in whole blood. Such cells are associated with metastatic breast cancer; however, the assay does not distinguish between post-mitotic cells shed by a tumor from clonogenic resting cells capable of seeding metastases. Accordingly, improved diagnostic methods are needed to detect circulating clonogenic cancerous cells.

### BRIEF SUMMARY OF THE INVENTION

The invention provides a method of identifying circulating clonogenic cancerous cells, specifically multiply-drug resistant (MDR) cancer stem cells. These and other advantages of the invention, as well as additional inventive features, will be apparent from the description of the invention provided herein.

### DETAILED DESCRIPTION OF THE INVENTION

In accordance with the inventive method, a fluid sample is withdrawn from a patient's circulation. The fluid sample can be, for example, blood, lymph, a leukapheresis product, or other circulating fluid. Any suitable volume of fluid can be withdrawn from the patient in accordance with the inventive method.

Following withdrawal of the fluid sample from the patient, it can be processed, if desired, to emich a cellular fraction. For example, a fluid sample of blood can be processed by leukapheresis to enrich the white cell fraction. Alternatively, a fluid sample can be processed to enrich a general cellular fraction, or another desired fraction of cells (e.g., red cells). Magnetic bead cancer stem cell enrichment and depletion of non-epithelial cells also enhances sensitivity. The fluid sample and/or cellular fraction can be further treated, as desired (e.g., by freezing, thawing, enzymatic treatment to remove contaminants such as nucleic acids, proteins, and the like, etc.).

The fluid sample and/or cellular fraction then is prepared for flow cytometry according to standard methods according to which single cells within the fluid sample and/or cellular fraction can be stained for identification or purification. However, preferably, the cells are first stained in bulk with anti-Ep-CAM SA-FITC (a/k/a human epithelial antigen (HEA) or epithelial specific antigen (ESA)), which facilitates separation using an immunomagnetic cell separation device such as an AUTOMACS device.

Preferably after the immunomagnetic cell separation device, in accordance with the inventive method, the single cells within the tissue sample are stained with dye-conjugated antibodies (preferably monoclonal antibodies) for identification or purification by flow cytometry. Preferably, the antibodies target CD45, CD90, CD117, CD133, and a marker of multiple drug resistance (such as ABCG2 (mitoxantrone resistance, Breast Cancer Resistance Protein 1), ABCB1 (MDR1, P-glycoprotein), ABCC1 (Multiple Resistance Protein) and Lung Resistance Protein (LRP)).

CD45 is preferably employed to remove hematopoetic-derived cells. However, other hematopoetic-specific antibodies could be used as functional equivalents. For example, a cocktail of lineage-specific antibodies can be employed to identify lineage-negative non-hematopoetic cells. Such lineage “cocktails” are composed of antibodies directed against epitopes expressed by RBC (red blood cells), lymphocytes of T-, B- and NK-lineages (CD3, CD4, CD8, CD19, CD16, CD56), monocytes, macrophages and histiocytes (tissue macrophages), eosinophills and basophills, neutrophills and granulocytes, platelets, and their precursors (non-epithelial lineage commitment).

The antibodies for use in the inventive method can be prepared by standard methodology and/or are commercially available (e.g., through Beckman-Coulter, Becton-Dickinson, Invitrogen and Chemicon. Dyes are purchased from Sigma and Invitrogen.).

Following labeling with the antibodies, the stained cells optionally are cultured in the presence of fluorescent MDR substrates. For example, Rhodainine 123 and Hoechst 33342 are substrates for the MDR transporters ABCG2 and ABCB1, respectively, and preferably the cells are exposed to both of these substrates. Other fluorescent MDR substrates can be employed as well (e.g., MDR Assays Using Acetoxymethyl Esters, Vybrant Multidrug Resistance Assay Kit, Diagnostic Assay for Multidrug Resistance, MDR Assays Using Glutathione-Reactive Probes, MDR Assays Using Mitochondrial Probes (e.g. R123), MDR Assays Using Nucleic Acid Stains (e.g. Hoechst 33342), BODIPY FL Verapamil, BODIPY Dihydropyridines, BODIPY FL Paclitaxel, BODIPY FL Vinblastine, BODIPY Prazosin and BODIPY Forskolin, MDR Assays Using Ion Indicators, and the like). Typically, the cells are exposed to these fluorescent MDR substrates for 15-90 min, but any suitable time can be employed.

Optionally, a viability dye can be added to the cells. Such dye can be, for example propidium iodide, DAPI, 7AAD, however, other suitable viability dyes can be used. Typically, viability dies act very quicldy. Thus, within a short period of time (at most, several minutes) following addition of the viability dye, the cells are subjected to flow cytometry. Preferably, the cells are subjected to the flow cytometry immediately after exposure to the viability dye.

Following the foregoing treatment with the antibodies, MDR substrates and/or viability dye(s), the cells are subjected to the flow cytometry. Where a viability dye is employed, preferably the flow cytometry is conducted as soon as possible, preferably immediately, after exposure to the viability dye. Flow cytometry can be conducted using standard methodology.

Following the flow cytometry, MDR cancer stem cells can be identified as having a combination of some of the following factors: 1) Live (viability dye excluding); 2) Singlet (by forward light scatter pulse analysis; 3) Non-hematopoietic (e.g., CD45 negative); 4) CD90, CD117, or CD133 positive; 6) MDR expression and/or activity (for example ABCG1+, ABCB1+, ABCC1+ and/or LRP+); 7) transport of the MDR fluorescent dye(s) (preferably excluding both Rhodamine 123 and/or Hoechst 33342). The MDR cancer stem cells also can be CD44+ and/or CD133−.

Another property of MDR cancer stem cells is for them to bear stem-cell associated markers and/or progenitor-cell associated markers. The principle feature employed to distinguish resting stem cells from progenitor cells is morphology. In single cell suspension, resting stem cells are small round cells, with high nucleus/cytoplasm ratio. This corresponds to low forward and side light scatter by flow cytometry. Both stem and progenitor populations express CD117+, CD90+ and/or CD133+. They also have scant RNA, as can be measured by flow cytometry using acridine orange staining. Progenitor cells are large metabolically active cells with low nucleus/cytoplasm ratio and can be found in disaggregated normal and neoplastic lung at a low frequency (<0.1%). Additionally direct analysis of morphologic features themselves (nucleus/cytoplasm ration and low-complexity morphology) by image analysis can distinguish between resting, self-protected stem cells and larger, mitotically active progenitor cells. MDR transporter expression and activity are quantified in both populations by detection of the specific transporter proteins, as discussed herein, and the transport of fluorescent substrates, respectively.

It will be observed that the presence of MDR cancer stem cells in the fluid sample obtained from the patient can support a diagnosis of metastasis cancer. Conversely, the absence of such cells in the fluid sample can be employed as a prognostic indicator suggesting that a known cancer within a patient has not metastasized, particularly when the assay is repeated.

### EXAMPLE

This example demonstrates the isolation and identification of MDR cancer stem cells.

A patient (stage IV breast cancer) had completed therapy consisting of high dose cyclophosphamide, etoposide and G-CSF. A leukapheresis product was collected from this patient upon rebound of peripheral counts for use in autologous transplantation and was cryopreserved.

Cells were thawed in DNAase (Pulmozyme), washed twice and incubated for 2 hrs on ice in medium containing 50% newborn calf serum, followed by a one-hour incubation with DNAase and 2-mercaptoethanol at room temperature. A total of 2.9×10⁹ viable cells were recovered from one transfer pack. 2×10⁹ cells were stained in bulk with anti-Ep-CAM SA-FITC (a/k/a human epithelial antigen (HEA) or epithelial specific antigen (ESA)) and separated on the AutoMACS immunomagnetic cell separation device. A total of 1.6×10⁶ cells (0.08% or 1/1,250 of input) was recovered from the AutoMACS column (set for maximal recovery at the expense of purity). These cells were stained for 7-color flow cytometry and analyzed on the Dako-Cytomation CyAn analyzer. Cells were acquired exhaustively at 12,000 events per second in less than 3 minutes.

ESA+ cells accounted for 75% of all stained cells within the low light scatter window. ESA+/CD45−/CD44+ cells (tumorigenic fraction) comprised 0.2% of input cells; and ESA+/CD45−/CD44+/ABCG2+ cells accounted for 0.03% of input cells. This population also expressed CD90 and CD117. The cells expressed the MDR transporter ABCG2 (breast cancer resistance protein) and stem cell markers in conjunction with the markers shown to define the tumorigenic-enriched fraction in a murine explant model.

### REFERENCES

Alonso L, Fuchs E. Stem cells of the skin epithelium. PNAS 100 (suppl 1): 11830-11835, 2003.
Amoh, Y., Li, L., Katsuoka, K., Penman, S., and Hoffman, R. M. Multipotent nestin-positive, keratin-negative hair-follicle bulge stem cells can form neurons. PNAS USA 102, 5530-5534, 2005.
Appelbaum F R, Fisher L D, Thomas E D. Chemotherapy v marrow transplantation for adults with acute nonlymphocytic leukemia: a five-year follow-up. Blood. 1988; 72:179-184.
Baines P, Visser J W. Analysis and separation of murine bone marrow stem cells by H33342 fluorescence-activated cell sorting. Exp Hematol. 1983; 11:701-708.
Biedler J L, Riehm H. Cellular resistance to actinomycin D in Chinese hamster cells in vitro: cross-resistance, radioautographic, and cytogenetic studies. Cancer Res. 1970; 30:1174-1184.
Blanpain, C., Lowry, W. E., Geoghegan, A., Polak, L., and Fuchs, E. Self-renewal, multipotency, and the existence of two cell populations within an epithelial stem cell niche. Cell 118, 635-648, 2004.
Bunting K D, Zhou S, Lu T, Sorrentino BP. Enforced P-glycoprotein pump function in murine bone marrow cells results in expansion of side population stem cells in vitro and repopulating cells in vivo. Blood. 2000; 96:902-909.
Burnett A K. Annotation: current controversies—which patients with acute myeloid leukaemia should receive a bone marrow transplantation? An adult treater's view. Br J Haematol. 2002; 118:357-364.
Carrion C, de Madariaga M A, Domingo J C. In vitro cytotoxic study of immunoliposomal doxorubicin targeted to human CD34(+) leukemic cells. Life Sci. 2004; 75:313-328.
Chen C C, Meadows B, Regis J, et al. Detection of in vivo P-glycoprotein inhibition by PSC 833 using Tc-99m sestamibi. Clin Cancer Res. 1997; 3:545-552.
Dey S, Ramachandra M, Pastan I, Gottesman M M, Ambudlcar S V. Evidence for two nonidentical drug-interaction sites in the human P-glycoprotein. Proc Natl Acad Sci U S A. 1997; 94:10594-10599.
Donnenberg A D, V S Donnenberg, H Shen. Rare-Event Detection and Analysis in Flow Cytometry. The Connection 5: 4-5, 20-21, 2003.
Donnenberg V S, and Donnenberg A D. Multiple drug resistance in cancer revisited: the cancer stem cell hypothesis. J Clin Pharmacol. 2005 August; 45(8):872-7.
Donnenberg V S, Burckart G J, Griffith B P, Jain A B, Zeevi A, Donnenberg A D. P-glycoprotein (P-gp) is Upregulated in Peripheral T-Cell Subsets from Solid Organ Transplant Recipients. Journal of Clinical Pharmacology, 2001; 41:1271-1279.
Donnenberg V S, Donnenberg A D, Thompson A W, Zeevi A, Burckart G J, Calhoun W J. In Vivo Maturation of Lung Dendritic Cells from BAL Following Segmental Antigen Challenge (SAC) in Asthmatic Patients. American Respiratory Alliance of Western Pennsylvania. The World Asthma Meeting, 2001.
Donnenberg V S, Donnenberg A D. Identification, rare-event detection and analysis of dendritic cell subsets in broncho-alveolar lavage fluid and peripheral blood by flow cytometry. Frontiers in Bioscience, 8:1175-1180, 2003.
Donnenberg V S. Burckart G J. Zeevi A. Griffith B P. Iacono A. McCurry K R. Wilson J W. Donnenberg A D. P-glycoprotein activity is decreased in CD4+ but not CD8+ lung allograft-infiltrating T cells during acute cellular rejection. Transplantation. 77(11):1699-706, 2004
Doyle L A, Yang W, Abruzzo L V, et al. A multidrug resistance transporter from human MCF-7 breast cancer cells. Proc Natl Acad Sci USA. 1998; 95:15665-15670.
Fiala S. The cancer cell as a stem cell unable to differentiate: a theory of carcinogenesis. Neoplasma. 1968; 15:607-622.
Fojo A, Hamilton T C, Young R C, Ozols R F. Multidrug resistance in ovarian cancer. Cancer. 1987; 60(suppl 8):2075-2080.
Galli, R., Binda, E., Orfanelli, U., Cipelletti, B., Gritti, A., De Vitis, S., Fiocco, R., Foroni, C., Dimeco, F., and Vescovi, A. Isolation and Characterization of Tumorigenic, Stem-like Neural Precursors from Human Glioblastoma. Cancer Res 64, 7011-7021, 2004;
Giangreco A, Shen H, Reynolds S D, Stripp B R. Molecular phenotype of airway side population cells. Am J Physiol Lung Cell Mol Physiol. 2004; 286:L624-L630.
Goodell M A, Brose K, Paradis G, Conner A S, Mulligan R C. Isolation and functional properties of murine hematopoietic stem cells that are replicating in vivo. J Exp Med. 1996; 183:1797-1806.
Gros P, Ben Neriah Y B, Croop J M, Housman D E. Isolation and expression of a complementary DNA that confers multidrug resistance. Nature. 1986; 323:728-731.
Gros P, Croop J, Roninson I, Varshavsky A, Housman D E. Isolation and characterization of DNA sequences amplified in multidrug-resistant hamster cells. Proc Natl Acad Sci USA. 1986; 83:337-341.
Hamburger A W, Salmon S E. Primary bioassay of human tumor stem cells. Science. 1977; 197:461-463.
Harker W G, Slade D L, Dalton W S, Meltzer P S, Trent J M. Multidrug resistance in mitoxantrone-selected HL-60 leukemia cells in the absence of P-glycoprotein overexpression. Cancer Res. 1989; 49:4542-4549.
Kessel D, Botterill V, Wodinsky I. Uptake and retention of daunomycin by mouse leukemic cells as factors in drug response. Cancer Res. 1968; 28:938-941.
Leonard G D, Fojo T, Bates S E. The role of ABC transporters in clinical practice. Oncologist. 2003; 8:411-424.
Ling V, Thompson L H. Reduced permeability in CHO cells as a mechanism of resistance to colchicine. J Cell Physiol. 1974; 83(1): 103-116.

Lum B L, Fisher G A, Brophy N A, et al. Clinical trials of modulation of multidrug resistance: pharmacolcinetic and pharmacodynamic considerations. Cancer. 72(suppl 11):3502-3514.
Merry S, Courtney E R, Fetherston C A, Kaye S B, Freshney R I. Circumvention of drug resistance in human non-small cell lung cancer in vitro by verapamil. Br J Cancer. 1987; 56:401-405.
Mulder A H, Visser J W. Separation and functional analysis of bone marrow cells separated by rhodamine-123 fluorescence. Exp Hematol. 1987; 15:99-104.
Niedernhofer L J. Odijk H. Budzowska M. van Drunen E. Maas A. Theil A F. de Wit J. Jaspers N G. Beverloo H B. Hoeijmakers J H. Kanaar R. The structure-specific endonuclease Ercc1-Xpf is required to resolve DNA interstrand cross-link-induced double-strand breaks. Molecular & Cellular Biology. 24(13):5776-87, 2004.
Prochazka M, Gaskins H R, Shultz L D, Leiter E H. The nonobese diabetic scid mouse: Model for spontaneous thymomagenesis associated with immunodeficiency (severe combined immunodeficiency mutation). Proc. Natl. Acad. Sci.-USA Vol. 89, pp. 3290-3294, 1992
Reya T, Morrison S J, Clarke M F, Weissman I L. Stem cells, cancer, and cancer stem cells. Nature. 2001; 414:105-111.
Roninson I B, Abelson H T, Housman D E, Howell N, Varshavslcy A. Amplification of specific DNA sequences correlates with multi-drug resistance in Chinese hamster cells. Nature. 1984; 309:626-628.
Roninson I B, Chin J E, Choi K G, et al. Isolation of human mdr DNA sequences amplified in multidrug-resistant KB carcinoma cells. Proc Natl Acad Sci USA. 1986; 83:4538-4542.
Rustum Y M, Radel S, Campbell J, Mayhew E. Approaches to overcome in vivo anti-cancer drug resistance. Prog Clin Biol Res. 1986; 223:187-202.
Shoemaker R H, Curt G A, Camey D N. Evidence for multidrug-resistant cells in human tumor cell populations. Cancer Treat Rep. 1983; 67:883-888.
Spangrude G J, Heimfeld S, Weissman I L. Purification and characterization of mouse hematopoietic stem cells. Science. 1988; 241: 58-62.
Tai, M.-H., Chang, C.-C., Olson, L. K., and Trosko, J. E. Oct4 expression in adult human stem cells: evidence in support of the stem cell theory of carcinogenesis. Carcinogenesis 26, 495-502, 2005.
Tan B, Piwnica-Worms D, Ratner L. Multidrug resistance transporters and modulation. Curr Opin Oncol. 2000; 12:450-458.
Twentyman P R, Fox N E, Bleehen N M. Drug resistance in human lung cancer cell lines: cross-resistance studies and effects of the calcium transport blocker, verapamil. Int J Radiat Oncol Biol Phys. 1986; 12:1355-1358.
Udomsakdi C, Eaves C J, Sutherland H J, Lansdorp P M. Separation of functionally distinct subpopulations of primitive human hematopoietic cells using rhodamine-123. Exp Hematol. 1991; 19:338-342.
Volm M. Multidrug resistance and its reversal. Anticancer Res. 1998; 18:2905-2917.
Weiden P L, Flournoy N, Thomas E D, et al. Antileukemic effect of graft-versus-host disease in human recipients of allogeneic-marrow grafts. N Engl J Med. 1979; 300:1068-1073.
Zhao, X., Das, A. V., Thoreson, W. B., James, J., Wattnem, T. E., Rodriguez-Sierra, J., and Ahmad, I., Adult corneal limbal epithelium: a model for studying neural potential of non-neural stem cells/progenitors. Dev Biol 250, 317-331, 2002.
Zhou S, Morris J J, Barnes Y, Lan L, Schuetz J D, Sorrentino B P. Bcrp1 gene expression is required for normal numbers of side population stem cells in mice, and confers relative protection to mitoxantrone in hematopoietic cells in vivo. Proc Natl Acad Sci USA. 2002; 99:12339-12344.
Zhou S, Schuetz J D, Bunting K D, et al. The ABC transporter Bcrp1/ABCG2 is expressed in a wide variety of stem cells and is a molecular determinant of the side-population phenotype. Nat Med. 2001; 7:1028-1034.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of identifying a circulating MDR cancer stem cell, the method comprising:
a. obtaining a fluid sample from a patient,
b. staining single cells from the fluid sample with dye-conjugated antibodies for identification or purification by flow cytometry, wherein the antibodies target a hemopoetic marker, CD90, CD117, or CD133, and a marker of multiple drug resistance,
c. optionally culturing the stained cells in the presence of one or more fluorescent MDR substrates,
d. optionally adding a viability dye to the cells,
e. subjecting the cells to flow cytometry;
whereby the MDR cancer stem cell is identified as having a plurality of the following factors: 1) Live (viability dye excluding); 2) Singlet (by forward light scatter pulse analysis; 3) Non-hematopoietic; +; 5) CD90, CD133, and/or CD117 positive; 6) MDR expression and/or activity by positive staining for the marker or multiple drug resistance and or transport of the fluorescent MDR substrate(s).

2. The method of claim 1, wherein the marker of multiple drug resistance is ABCG2 (mitoxantrone resistance, Breast Cancer Resistance Protein 1), ABCB1 (MDR1, P-glycoprotein), ABCC1 (Multiple Resistance Protein) or Lung Resistance Protein (LRP).

3. The method of claim 1, wherein the hemopoetic marker is CD45.

4. The method of claim 2, wherein the hemopoetic marker is CD45.

5. The method of claim 1, wherein a fluorescent MDR substrate is Rhodamine 123 and/or Hoechst 33342.

6. The method of claim 2, wherein a fluorescent MDR substrate is Rhodamine 123 and/or Hoechst 33342.

7. The method of claim 3, wherein a fluorescent MDR substrate is Rhodamine 123 and/or Hoechst 33342.

8. The method of claim 4, wherein a fluorescent MDR substrate is Rhodamine 123 and/or Hoechst 33342.

9. A method of identifying a circulating MDR cancer stem cell, the method comprising:
a. obtaining a fluid sample from a patient,
b. identifying a fraction of cells from within the fluid sample that are epithelium-specific antigen (ESA+), and
c. staining single ESA+ cells from the fluid sample with dye-conjugated antibodies for identification or purification by flow cytometry, wherein the antibodies target a hemopoetic marker, CD90, CD117, or CD133, and a marker of multiple drug resistance,
d. optionally culturing the stained cells in the presence of one or more fluorescent MDR substrates,
e. optionally adding a viability dye to the cells,
f. subjecting the cells to flow cytometry;
whereby the MDR cancer stem cell is identified as having a plurality of the following factors: 1) Live (viability dye excluding); 2) Singlet (by forward light scatter pulse analysis; 3) Non-hematopoietic; +; 5) CD90, CD133, and/or CD117 positive; 6) MDR expression and/or activity by positive staining for the marker or multiple drug resistance and or transport of the fluorescent MDR substrate(s).

10. The method of claim 9, wherein the ESA+ cells are identified by staining the cells within the fluid sample with anti-Ep-CAM SA-FITC.

11. The method of claim 10, wherein following staining with anti-Ep-CAM SA-FITC, the cells are subjected to separation using an immunomagnetic cell separation device.

12. The method of claim 9, wherein the marker of multiple drug resistance is ABCG2 (mitoxantrone resistance, Breast Cancer Resistance Protein 1), ABCB1 (MDR1, P-glycoprotein), ABCC1 (Multiple Resistance Protein) or Lung Resistance Protein (LRP).

13. The method of claim 9, wherein the hemopoetic marker is CD45.

14. The method of claim 9, wherein a fluorescent MDR substrate is Rhodamine 123 and/or Hoechst 33342.
